(19) United States
(12) Patent Application Publication
(10) Pub. No.: US 2021/0002207 A1
(43) Pub. Date: Jan. 7, 2021

(54) A process for the preparation of Vigabatrin

(71) Applicant: Aurobindo Pharma Ltd., Hyderabad (IN)

(72) Inventors: Senthil Kumar NATARAJAN, Hyderabad (IN); Karumanchi KISHORE, Hyderabad (IN); Gadde SUNIL, Hyderabad (IN); Krishna VANCHANAGIRI, Hyderabad (IN); V.r.moturu KRISHNAMURTHY, Hyderabad (IN); Sivakumaran MEENAKSHISUNDERAM, Hyderabad (IN)

(21) Appl. No.: 16/982,548

(22) PCT Filed: Mar. 13, 2019
(86) PCT No.: PCT/IB2019/052027

§ 371 Date: Sep. 19, 2020

(30) Foreign Application Priority Data
Mar. 20, 2018 (IN) 201841010221

(51) Int. Cl. C07C 227/40

(52) U.S. Cl. CPC C07C 227/40

(57) ABSTRACT

The present invention provides a process for the preparation of vigabatrin of formula (I) comprising of dissolving vigabatrin in water, optionally treating with charcoal, filtering and adding an acid to the reaction mass followed by the addition of an organic solvent and then isolating vigabatrin of formula (I) with high purity.

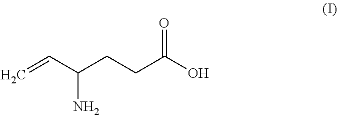

### FIELD OF THE INVENTION

The present invention relates to a process for the preparation of vigabatrin of formula (I) with high purity.

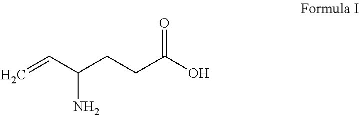

### BACKGROUND OF THE INVENTION

Vigabatrin is chemically known as 4-aminohex-5-enoic acid and is marketed under the brand name Sabril®. Vigabatrin is a well-known anti-epileptic agent and acts as an irreversible inhibitor of gamma-aminobutyric acid transaminase (GABA-T) the enzyme responsible for the catabolism of the inhibitory neurotransmitter gamma-aminobutyric acid (GABA) in the brain.

Vigabatrin also known as “vinyl-GABA” was first disclosed in U.S. Pat. No. 3,960,927 which discloses a process for the preparation of 4-amino hex-5-enoic acid (vigabatrin) by the reduction of 4-amino-5-yne-hexanoic acid of formula (II) with Lindlar catalyst (5% Pd/CaCO₃/PbO₂) in the presence of pyridine and hydrogen atmosphere.

The process is depicted in below scheme:

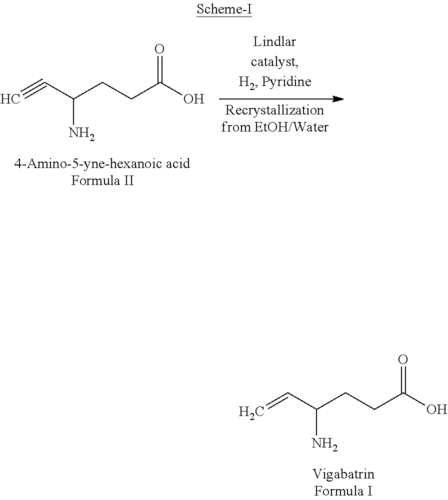

U.S. Pat. No. 4,178,463 discloses a process for the preparation of vigabatrin of formula (I), comprising the reaction of 1,4-dichloro-2-butene (V) with diethyl malonate under basic conditions to produce 2-vinyl cyclopropane-1,1-diethyldicarboxylate (IV) which is then reacted with ammonia under pressure to form 3-carboxamido-5-vinyl-2-pyrrolidone (III), which is further hydrolyzed under acidic conditions to form 4-amino-5-hexenoic acid (I).

The process is depicted in below scheme:

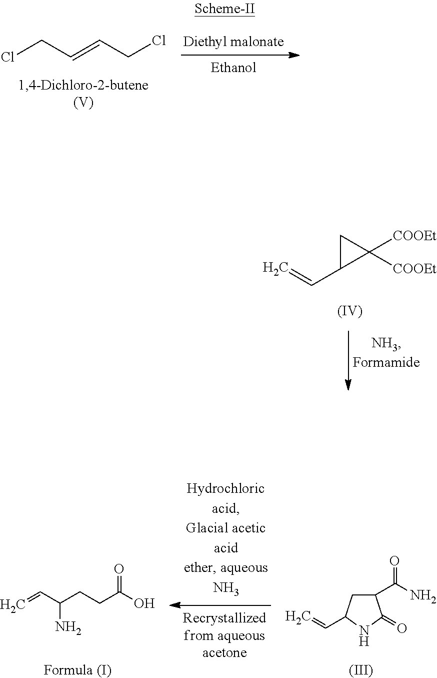

US 20130165693 A1 discloses the reduction of succinimide with sodium borohydride in ethanol to obtain 5-ethoxy-2-pyrrolidone (VII) followed by treating with vinyl magnesium chloride in THF optionally in the presence of base to obtain 5-Vinyl-2-pyrrolidone (VI) which on further hydrolysis with acid or base affords vigabatrin (I).

The process is shown in below scheme:

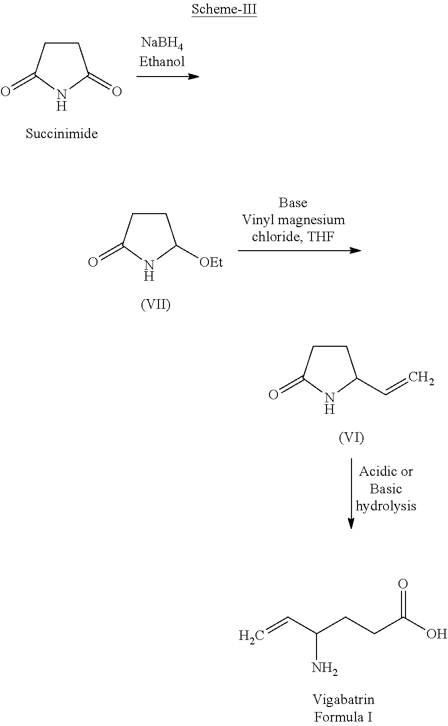

The major drawback associated with the above mentioned prior-art processes is the formation of unwanted impurity in vigabatrin drug product which are not easy to remove by conventional purification or crystallization methods.

Hence, there is a need of an improved process for the preparation of vigabatrin of formula (I) which devoid the disadvantage of the prior art processes as mentioned herein above and gives vigabatrin of formula (I) with high purity. The process of the present invention describes a purification process using specific solvent system which results vigabatrin of formula (I) with high purity.

### OBJECTIVE OF THE INVENTION

The objective of the present invention is to provide an industrially viable process for the preparation of vigabatrin of formula (I) with high purity and good yield.

### SUMMARY OF THE INVENTION

In an embodiment, the present invention provides a process for the preparation of vigabatrin of formula (I) with high purity, which comprises the steps of:

a. preparing a solution of vigabatrin in water;

b. optionally treating with charcoal and filtering the solution of step (a);

c. adding an acid to the solution of step (a) or (b);

d. adding an organic solvent;

e. isolating vigabatrin of Formula (I).

### DETAILED DESCRIPTION OF THE INVENTION

The present invention relates to a process for the preparation of vigabatrin of formula (I) by dissolving vigabatrin in water, optionally treating with charcoal, filtering and adding an acid to the reaction mass followed by adding an organic solvent and isolating vigabatrin of formula (I).

Vigabatrin used in the present invention is prepared by using the process disclosed in the present invention or by prior-art processes.

The acid used in step (c) comprises of an organic acid selected from but not limited to formic acid, acetic acid, oxalic acid, propanoic acid, lactic acid, maleic acid, citric acid, valeric acid, benzoic acid or mixtures thereof or an inorganic acid selected from but not limited to hydrochloric acid, sulphuric acid, phosphoric acid, nitric acid or mixtures thereof.

The organic solvent of step (d) is selected from but not limited to a polar protic solvent comprises methanol, ethanol, isopropanol, n-butanol, acetic acid and/or mixtures thereof a polar aprotic solvent comprises dimethylformamide (DMF), dimethylsulfoxide (DMSO), tetrahydrofuran (THF), acetonitrile, acetone, ethyl acetate, N-methyl pyrrolidone and/or mixture thereof; and a non-polar solvents comprises hexane, benzene, toluene, 1,4-dioxane, chloroform, diethyl ether, dichloromethane (CH₂Cl₂) or mixture thereof.

Isolation of vigabatrin of step (e) is carried out by cooling the reaction mixture of step (d) to obtain vigabatrin of formula (I).

### Example-1

### Preparation of (RS)-5-Ethoxy-2-Pyrrolidinone

Succinimide (150 grams) was suspended in ethanol (2250 ml) and cooled to −10° C. to −5° C. Sodium borohydride (86 grams) and hydrogen chloride in ethanol (1020 ml) were added to the above suspension at −5° C. to 5° C. and stirred at the same temperature for 6 hrs. Then the reaction mass pH was adjusted to 8.0-8.5 by adding potassium hydroxide in ethanol and concentrated completely under reduced pressure. To the resulting residue, methylene chloride (3000 ml) and water (600 ml) were added and separated. The organic layer was concentrated completely and cyclohexane (450 ml) was added and heated to 35-40° C. The resulting solution was cooled to 20-30° C., filtered and dried to yield (RS)-5-ethoxy-2-pyrrolidinone.

Yield: 157.5 grams

### Example-2

### Preparation of Vigabatrin

To a solution of (RS)-5-Ethoxy-2-pyrrolidinone (100 grams) in THF (150 ml), 2,6-di-tert-butyl-4-methylphenol (BHT, 1 g) was added and cooled to −15 to −10° C. ethyl magnesium bromide (310 ml, 2M in THF) and vinyl magnesium bromide solution (1162 ml, 1M in THF, 1.5 mole equivalents) were added to the reaction mass at −15° C. to −10° C. and stirred for 30 min. The reaction mass was heated to reflux for 1 hour. Thereafter, the reaction mass was cooled to 0-5° C. and added water (1000 ml). The pH of the reaction mass was adjusted to 6.0-6.5 with acetic acid and extracted with methylene chloride (2000 ml) and the organic layer was separated. The resulting organic layer was concentrated completely to yield (RS)-5-vinyl-2-pyrrolidinone as an oily mass. Aqueous potassium hydroxide solution was added to the above oily mass and heated to 90-100° C. for 3 hours. Thereafter, the reaction mass was cooled to 25-35° C. and isopropyl alcohol (525 ml) was added followed by acetic acid and stirred for 3 hours. The obtained slurry was cooled to 0-5° C. and maintained for 2 hours. The product was filtered, washed with isopropyl alcohol and dried at 50-60° C. under reduced pressure to obtain vigabatrin crude.

Yield: 46 grams

### Example-3

### Purification of Vigabatrin

Vigabatrin crude (31 grams) was dissolved in water (124 ml) and heated to 40-45° C. The solution was treated with charcoal, filtered and washed with water (31 ml). The filtrate was concentrated and acetic acid (4.7 ml) was added at 70° C. The solution was cooled to 50-60° C. and isopropyl alcohol (310 ml) was added. The slurry was further cooled to 20-30° C. and stirred for 1 hour followed by cooling to 0-5° C. The obtained vigabatrin was filtered and washed with isopropyl alcohol. The crude product was dried at 50-60° C. under reduced pressure to obtain pure vigabatrin.

Yield: 28.5 grams

Purity by HPLC: 99.58%

### Example-4

### Purification of Vigabatrin

Vigabatrin crude (50 grams) was added to water (50 ml) at 20-30° C. and to the slurry, acetic acid (7.5 ml) was added, heated to 65-70° C. and stirred for 30 minutes. The resulting solution was cooled to 50-60° C. and isopropyl alcohol (500 ml) was added. The slurry was cooled to 20-30° C. and stirred for 1 hour. The obtained vigabatrin was filtered, washed with isopropyl alcohol and dried at 50-60° C. under reduced pressure to obtain pure vigabatrin.

Yield: 48 grams

Purity by HPLC: 99.82%

### Example-5

### Purification of Vigabatrin

Vigabatrin crude (100 grams) was added to water (150 ml) at 20-30° C. and to the slurry, acetic acid (25 ml) was added, heated to 65-70° C. and stirred for 30 minutes. The resulting solution was cooled to 50-60° C. and isopropyl alcohol (750 ml) was added. The slurry was cooled to 20-30° C. and stirred for 1 hour followed by cooling to 0-5° C. The obtained product was filtered, washed with isopropyl alcohol and dried at 50-60° C. under reduced pressure to obtain pure vigabatrin.

Yield: 90 grams

Purity by HPLC: 99.97%

We claim:

1. A process for the preparation of vigabatrin of formula (I), which comprises the steps of:
a) preparing a solution of vigabatrin in water;
b) optionally treating with charcoal and filtering the solution of step (a);
c) adding an acid to the solution of step (a) or (b);
d) adding an organic solvent;
e) isolating vigabatrin of Formula (I).

2. The process according to claim 1, wherein the acid used in step (c) is selected from an organic acid or an inorganic acid.

3. The process according to claims 1 and 2, wherein the organic acid is selected from formic acid, acetic acid, oxalic acid, propanoic acid, lactic acid, maleic acid, citric acid, valeric acid, benzoic acid or mixtures thereof and the inorganic acid is selected from hydrochloric acid, sulphuric acid, phosphoric acid, nitric acid or mixtures thereof.

4. The process according to claim 1, wherein the organic solvent of step (d) is selected from methanol, ethanol, isopropanol, n-butanol, acetic acid, dimethylformamide, dimethylsulfoxide, tetrahydrofuran, acetonitrile, acetone, ethyl acetate, N-methyl pyrrolidine, hexane, benzene, toluene, 1,4-dioxane, chloroform, diethyl ether, dichloromethane or mixtures thereof.

5. The process according to claim 1, wherein the isolation of step (d) is carried out by cooling the reaction mixture.
